(19) United States
(12) Patent Application Publication
(10) Pub. No.: US 2008/0097474 A1
(43) Pub. Date: Apr. 24, 2008

(54) METHOD OF PERFORATING A BIOLOGICAL MEMBRANE

(75) Inventors: Eric G. Heegaard, Saint Paul, MN (US); Roger W. Heegaard, Minneapolis, MN (US); John K. Lampe, Saint Paul, MN (US)

Correspondence Address: SHERRILL LAW OFFICES, 4756 BANNING AVE, SUITE 212, WHITE BEAR LAKE, MN, 55110-3205, US

(73) Assignee: MEDTREO, LLC, Minneapolis, MN (US)

(21) Appl. No.: 11/960,065

(22) Filed: Dec. 19, 2007

Related U.S. Application Data
(63) Continuation of application No. 11/032,906, filed on Jan. 11, 2005.
(60) Provisional application No. 60/535,432, filed on Jan. 12, 2004.

(51) Int. Cl. A61B 17/42

(52) U.S. Cl. 606/125

(57) ABSTRACT

Perforating a biological membrane by (i) obtaining a medical device having a longitudinally elongated shaft and a blunt tipped contact face disposed at the longitudinal distal end of the shaft, and (ii) concurrently pressing the contact face against a biological membrane and rotating the shaft about the longitudinal axis so as to effect rotation of the contact face and the biological membrane about a longitudinal centerpoint on the contact face until the membrane is perforated.

This is a continuation application of U.S. patent application Ser. No. 11/032,906, filed Jan. 11, 2005, which claims the benefit of U.S. Provisional Patent Application Ser. No. 60/535,432 filed on Jan. 12, 2004.

### FIELD OF INVENTION

The present invention generally relates to a medical device for perforating membranes such as the amniotic membrane of a pregnant woman in order to facilitate birth. Specifically, the device is an elongated instrument with a contact face on a distal end. The user inserts the device into the vagina of a pregnant woman. The user places the contact face of the device against the membrane. In accordance with at least one embodiment, the user rotates the device around the device's longitudinal axis. Projections on the contact face at the perforator's distal end rupture the membrane by shearing or cutting the membrane. The rupturing of the membrane releases the amniotic fluid which can facilitate the birth of the baby.

### BACKGROUND

For a human baby to be born, the amniotic membrane must rupture and release the amniotic fluid. The escape of amniotic fluid enhances uterine contractions. Frequently, a rupture occurs without intervention. However, in many cases, the attending physician must take action to rupture the membrane.

Although generally a straightforward procedure, rupturing the amniotic sac can, in some instances, become a fairly difficult procedure. Reaching the amniotic sac requires access through the vagina. The tissue in the vagina is sensitive, and therefore some care is required to avoid bleeding and bruising of the tissue. The doctor must work in the confined space of the vagina. The doctor cannot see the cervix or the amniotic sac and therefore must work by touch. Fat tissue can complicate matters. Moreover, in some cases, insufficient dilation of the cervix may limit access to the amniotic membrane. Finally, the position of the cervix and the location of the fetus in the amniotic sac can affect the ease with which the procedure can be performed.

The prior art contains many instruments for rupturing the amniotic membrane. U.S. Pat. Nos. 3,624,747 and 3,533,411 both to McKnight et al. teach an instrument in common use. Hollister Incorporated distributes the device under the trademark Amnihook.®.

This device, like many on the market, resembles a crochet hook. The Amnihook® has an elongated shaft with a small hook on a narrower, distal end. The curvature of the hook forms a blunt tip with a hook on one side. To use the Amnihook®, a doctor guides the distal end of the instrument into the vagina with two fingers. The doctor can protect the tissue of the vagina by burying the hooked side between the two fingers. The tip is guided through the cervix. Once the doctor has positioned the blunt side against the amniotic membrane, the doctor rotates the tool ninety degrees to bring the hooked end in contact with the amniotic membrane. By using a pulling action, the doctor can snag the membrane with the hook and rupture amniotic sac.

Although still widely used in the practice of obstetrics, the Amnihook® has recognized shortcomings. First, placing the Amnihook® in the proper position to perform the procedure can be difficult. The shaft of the device is straight and relatively inflexible. The vagina is generally not straight, and the shape varies among woman. Guiding the device through the vagina without damaging tissue requires care. Moreover, the Amnihook® must be positioned at the correct angle for it to snag the amniotic membrane. This may require the doctor to repeatedly adjust the position of the device in the vagina. These adjustments can prolong the procedure and can cause pain to the patient. Moreover, the increased number of manipulations can increase the likelihood of injury from the hook to the tissue of the patient or to the fetus.

Second, once the device is in the proper position, the action of hooking the membrane can be difficult. The smooth membrane may prevent the hooking of the membrane. This is particularly true if the hook is slightly out-of-position. Pulling the hook in the proper direction may prove difficult given the confined space and the anatomy of the mother and the fetus.

Other devices in the prior art have tried to offer improvements to the Amnihook.® However, these devices also have shortcomings. For example, U.S. Pat. No. 5,968,055 to Dimitriu describes a device that is also based on the “crochet hook” principle. The device taught in the Dimitriu patent differs slightly from the Amnihook®. These differences include a curvature of the shaft on the end opposite the hook; a flat surface opposite the hook for resting the index finger; and a rounded “I-beam” shaped shaft. The main purpose of these improvements is to improve the stability and controllability of the device. However, these improvements make the device more inflexible. This inflexibility makes the device less capable of dealing with variations in anatomy.

U.S. Pat. No. 5,846,250 to Parker, III, describes another instrument based on the crochet hook principle. The patent to Parker, III, differs in that it teaches an elongated shaft with a “flexing portion.” The device can bend more readily at the “flexing portion.” However, the device in many applications could suffer from the instability and lack of control that devices such as the one described in the Dimitriu patent were intended to correct.

Many other devices employ the “crochet hook” design. These devices suffer from many of the same shortcomings of those described above.

Other devices depart from the “crochet hook” design. For example, U.S. Pat. No. 4,662,376 to Belanger reveals a device that uses suction to pull amniotic membrane into a tube. “Piercing pins” inside the tube then cut the membrane thereby rupturing the amniotic sac. This device is more complicated in design and therefore would likely be more costly to manufacture. In addition, maintaining the suction to perform the cutting operation may be difficult given variations in anatomy. Finally, the device could require a larger opening within which to operate. Especially in instances where the cervix has not dilated sufficiently or the position of the cervix makes access through it difficult, use of such a device may be foreclosed.

Another device that departs from the “crochet hook” design is the Arom-Cot™ from Utah Medical Products. This device is a finger cot with a hook attached at the tip. More specifically, the Arom-Cot™ is a latex sleeve that fits over a single finger. A small plastic hook is attached to the latex near the tip of the finger. The hook is positioned such that the tip of the hook points toward the bottom of the finger. When the finger with the Arom-Cot™ is inserted into the vagina, the position of the hook reduces the likelihood of the hook snagging tissue. Once the finger with Arom-Cot™ is touching the amniotic sac, the doctor can draw the finger and the hook across the surface of the amniotic membrane to rupture it.

The Arom-Cot™ also suffers from deficiencies. First, putting the cot on and taking it off the finger may be difficult and time-consuming. This is particularly true if the hand already has a latex glove on it. Second, the Arom-Cot™ may not properly fit the wide range of finger sizes of potential users. Finally, extracting the finger with the Arom-Cot without damaging tissue can be difficult.

Accordingly, a continuing need exists for a safe, inexpensive and easy-to-use tool for rupturing the amniotic membrane.

### SUMMARY OF THE INVENTION

The invention is a medical device and method of perforating a biological membrane with the medical device. The medical device includes a longitudinally elongated shaft with a contact face disposed at one end of the shaft effective for perforating a biological membrane when the contact face is pressed against the biological membrane and rotated about the longitudinal axis.

The medical device can be used to perforate a biological membrane by concurrently pressing the contact face against a biological membrane and rotating the shaft about the longitudinal axis so as to effect rotation of the contact face and the biological membrane about a longitudinal centerpoint on the contact face until the membrane is perforated. A specific application is use of the medical device to encourage childbirth by inserting the device into the vagina of a pregnant woman, placing the contact face of the device, or projections longitudinally extending from the contact face of the device, against the amniotic membrane, and rotating the device about the device's longitudinal axis until the membrane is perforated.

### BRIEF DESCRIPTION OF THE DRAWINGS

FIG. 1A is a perspective view of one embodiment of a membrane perforator.

FIG. 1B is an enlarged perspective view of the contact surface of the membrane perforator shown in FIG. 1A.

FIG. 1C is a perspective view of the membrane perforator shown in FIG. 1A being used to perforate the amniotic membrane of a pregnant woman.

FIG. 1D is an enlarged perspective view of the membrane perforator shown in FIG. 1A being rotated to perforate a biological membrane.

FIG. 1E is a perspective view of the membrane perforator shown in FIG. 1A being bent during use to perforate the amniotic membrane of a pregnant woman.

FIG. 1F is a perspective view of the membrane perforator shown in FIG. 1A being used at an angle to the surface of an amniotic membrane to perforate the amniotic membrane.

FIG. 1G is an enlarged side view of the membrane perforator shown in FIG. 1A held at an angle and rotated to perforate a biological membrane.

FIG. 1H is an enlarged side view of the membrane perforator shown in FIG. 1A held at an angle and pulled to perforate a biological membrane.

FIG. 2A is an enlarged perspective view of the contact surface of a second embodiment of a membrane perforator.

FIG. 2B is an enlarged side view of the membrane perforator shown in FIG. 2A pushed into a biological membrane in order to pierce the membrane.

FIG. 3 is an enlarged perspective view of the contact surface of a third embodiment of a membrane perforator.

FIG. 4 is an enlarged perspective view of the contact surface of a fourth embodiment of a membrane perforator.

FIG. 5 is an enlarged perspective view of the contact surface of a fifth embodiment of a membrane perforator.

FIG. 6 is an enlarged perspective view of the contact surface of a sixth embodiment of a membrane perforator.

FIG. 7 is an enlarged perspective view of the contact surface of a seventh embodiment of a membrane perforator.

FIG. 8 is an enlarged perspective view of the contact surface of an eighth embodiment of a membrane perforator.

### DETAILED DESCRIPTION OF THE INVENTION INCLUDING A BEST MODE

Nomenclature

100 medical device or membrane perforator (1st embodiment)

101 shaft

102 contact face

103 distal end

104 projections

105 sharp edges

106 first hand

107 second hand

109 index finger

110 middle finger

111 vagina

112 cervix

113 amniotic sac

114 amniotic membrane

115 fetus

116 pressure toward distal end

117 direction of rotation

118 ripples on membrane

119 bentshaft

120 direction of drag

200 medical device or membrane perforator (2^{nd} embodiment)

202 contact face

203 distal end

204 projections

214 amniotic membrane

216 longitudinal direction

300 medical device or membrane perforator (3rd embodiment)

302 contact face

303 distal end

304 projections

305 sharp edges

400 medical device or membrane perforator (4^{th} embodiment)

402 contact face

403 distal end

404 projections

500 medical device or membrane perforator (5^{th} embodiment)

502 contact face

503 distal end

504 projections

600 medical device or membrane perforator (6^{th} embodiment)

602 contact face

603 distal end

604 projections

627 serrations

700 medical device or membrane perforator (7^{th} embodiment)

702 contact face

703 distal end

704 projections

730 cap

800 medical device or membrane perforator (8^{th} embodiment)

802 contact face

803 distal end

804 center projection

804 a cutter projections

835 counter-clockwise rotation

Construction

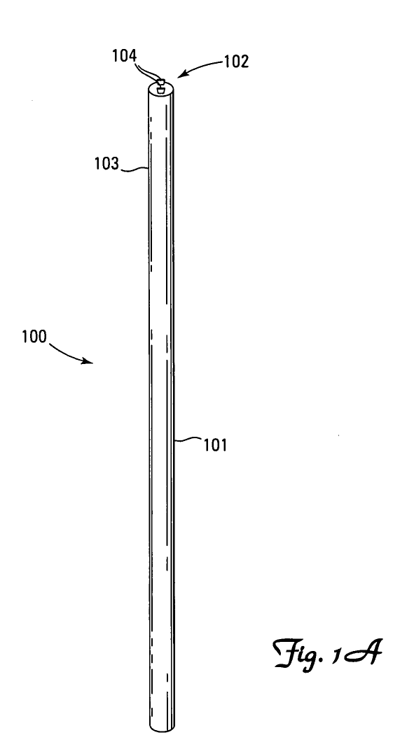

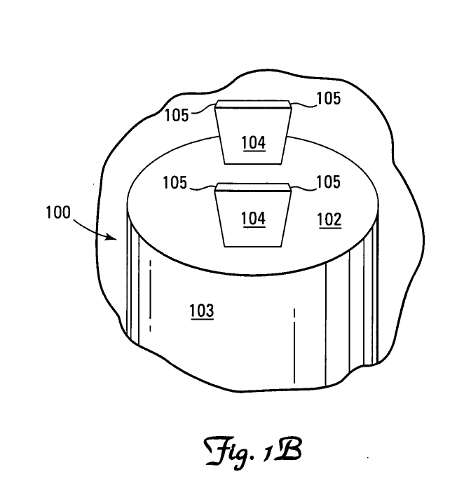

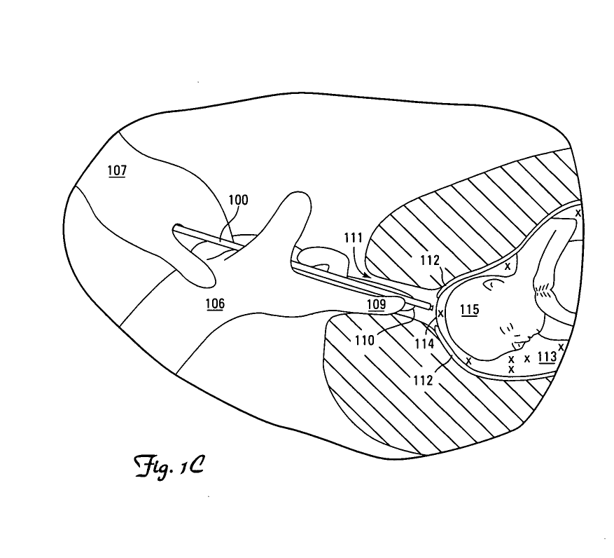

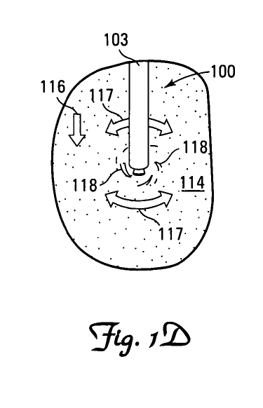

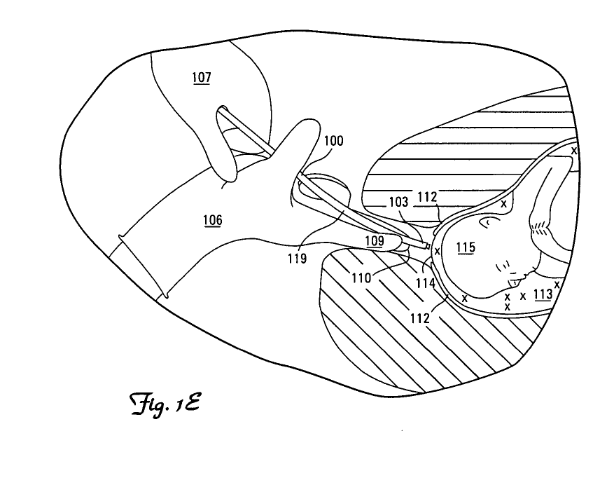

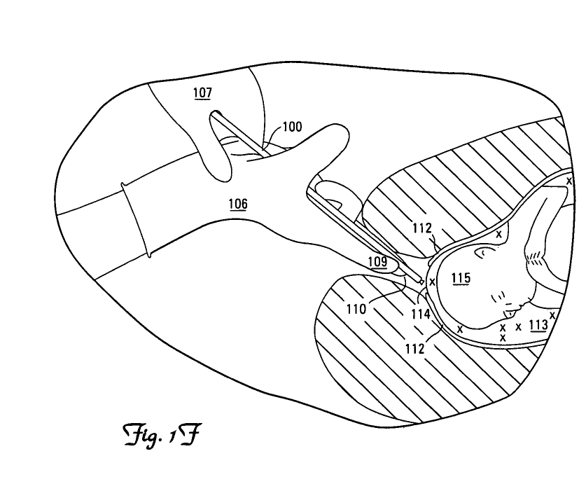

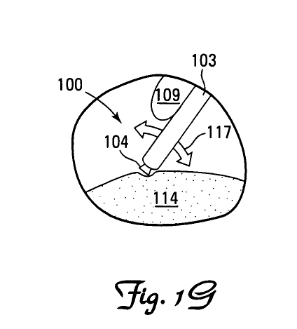

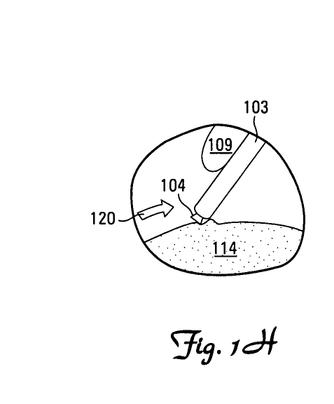

Referring generally to FIG. 1A, the invention is a medical device 100 which is (i) simple and easy to manufacture, (ii) simple and easy to use, and (iii) can quickly, easily and safely perforate the amniotic membrane 114 of the amniotic sac 113 for purposes of facilitating childbirth even when only a small area of the amniotic membrane 114 is accessible.

The medical device 100 can be manufactured as a disposable tool (e.g., manufactured from plastic) or a reusable tool (e.g., manufactured of medical grade stainless steel).

### First Embodiment

FIGS. 1A-1F show a membrane perforator 100 according to the first embodiment of the invention. The membrane perforator 100 shown in FIGS. 1A-1F consists of a shaft 101, a contact face 102 on the distal end 103 of the shaft 101, and projections 104 on the contact face 102. FIG. 1B shows a close up view of the projections 104 on the contact face 102. In this embodiment, the projections 104 are narrower at their base. The tops and the sides of the projections 104 could form relatively sharp edges 105.

The shaft 101 can be rigid or flexible, with the degree of flexibility variable from a highly flexible nearly limp shaft 101 to a moderately flexible stiff shaft 101.

The membrane perforator 100 could be of varying lengths. A length of between ten to twelve inches could be suitable for many applications. The membrane perforator 100 could be made of a variety of materials. A plastic material that could be sterilized and packaged in a sterilized condition could be suitable for many applications.

FIGS. 1C-1F show how the membrane perforator 100 according to the first embodiment could work. FIG. 1C shows first and second hands, 106 and 107, of a user holding the membrane perforator 100. The user's hands, 106 and 107, have already guided the membrane perforator 100 through the vagina 111 and positioned the contact face 102 against that portion of the amniotic sac 113 exposed in the dilated cervix 112. The user could guide the membrane perforator 100 into this position using the same procedure commonly used with other devices. Using this procedure, the user could guide the distal end 103 of the membrane perforator 100 through the vagina 111 with the contact face 102 and its projections 104 positioned between the index and middle fingers, 109 and 110. This position prevents unintended contact between the contact face 102 of the membrane perforator 100 and the sensitive tissue of the vagina 111.

With the membrane perforator 100 in the position shown in FIG. 1C, the user could use a first hand 106 and the index and middle fingers, 109 and 110, to maintain the membrane perforator 100 in the desired position. The second hand 107 could be used to lightly press the distal end 103 of the membrane perforator 100 against the amniotic membrane 114 and simultaneously rotate 117 the shaft 101 around its longitudinal axis. As shown in FIG. 1D, this application of light forward pressure 116 and rotation 117 causes the amniotic membrane 114 to form ripples 118 on the amniotic membrane 114 and weaken under the shearing stress. The ripples 118 make it easier for the sharp edges 105 on the projections 104 to cut into the amniotic membrane 114 and rupture the amniotic sac 113.

For most applications, the rotation 117 and forward pressure 116 would not have to be significant. One partial rotation 117 of ten to thirty degrees could be sufficient to pierce the amniotic membrane 114. If one partial rotation 117 in one direction were insufficient, the perforator could be rotated 117 approximately the same amount in the opposite direction. This process could be repeated until the amniotic sac 113 is perforated.

The user could vary the position and orientation of the membrane perforator 100 to best accomplish the task of perforating the amniotic membrane 114. For example, in some instance it might be preferable hold the shaft 101 of the membrane perforator 100 in an unbent position and in a roughly perpendicular position to the surface of the amniotic membrane 114. FIGS. 1C and 1D show the entire shaft 101 of the membrane perforator 100 held in an unbent position and in a roughly perpendicular position in relation to the amniotic membrane 114.

In other situations it may be preferable to bend the shaft 101 of the membrane perforator 100 to accommodate, for example, the anatomy of a patient's vagina 111 or the position of the fetus 115. For such situations the shaft 101 could be made sufficiently flexible to be bent somewhat by the user. FIG. 1E shows the membrane perforator 100 being bent somewhat in the hands 106 and 107, of the user. By bending the shaft 101 the user creates a bent shaft 119 which allows the user to hold the distal end 103 of the membrane perforator 100 in a roughly perpendicular position in relation to the surface of the amniotic membrane 114. This would permit fuller engagement between the two projections 104 and the amniotic membrane 114. Yet, even with this bending of the shaft 101 of the perforator 100, the user could produce sufficient torque to rotate the shaft 101 in rotational direction 117 and bite into the surface of the amniotic membrane 114 to rupture the amniotic sac 113 as shown in FIG. 1D.

Finally, in some situations it may be preferable to hold the membrane perforator 100 at an angle in relation to the amniotic membrane 114. FIG. 1F shows a user's hands, 106 and 107, holding the membrane perforator 100 at an angle to the surface of the amniotic membrane 114. FIG. 1G shows a close-up of the distal end 103 of the membrane perforator 100 held in approximately this same angled position. Even at this angle, the perforator 100 could be rotated as shown in FIG. 1G in rotational direction 117, and the edges 105 of one or both of the projections 104 could snag the amniotic membrane 114. Alternatively, if space allowed, the user could simply drag the projections 104 across the amniotic membrane 114 in direction 120 to pierce it as shown in FIG. 1H.

Rupturing the amniotic sac 113 with the membrane perforator 100 and in the ways shown in FIGS. 1A-1H has several advantages over the instruments and methods used in the prior art. First, the method allows the user to work in very confined spaces because the movements needed to pierce the amniotic sac 113 could be very slight and localized. Second, the membrane perforator 100 does not require precise positioning in relation to the amniotic sac 113 in order to work. The membrane perforator 100 can rupture the amniotic sac 113 with different motions including rotational, pulling, or pushing motions, and from different angles. Third, the flexibility of the membrane perforator 100 allows the membrane perforator 100 to be bent to work around parts of anatomy that other devices could not. Finally, despite the device's flexibility, sufficient force could be imparted to the projections 104 to pierce the amniotic membrane 114.

### Second Embodiment

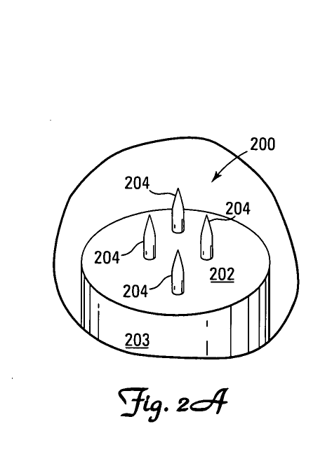

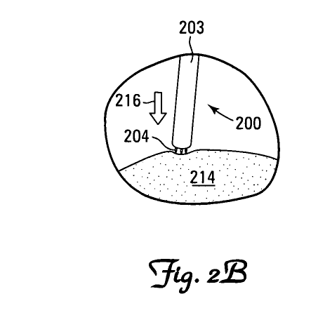

FIGS. 2A and 2B show a partial view of a membrane perforator 200 according to a second embodiment of the invention. The key difference between this membrane perforator 200 and the first embodiment of the membrane perforator 100 shown above concerns the projections 204. In the second embodiment the shape of the projections 204 generally resemble cones with somewhat sharp tips. One advantage of projections 204 such as these are that the membrane perforator 200 could more readily be used to puncture an amniotic membrane 214 by applying pressure in a longitudinal direction 216 toward the distal end 203 as shown in FIG. 2B. In addition, the rotating and dragging methods described above could also be used (not shown in relation to this embodiment).

This embodiment ostensibly has the disadvantage of having exposed points on the projections 204 that could injure tissue or the fetus (not shown in relation to this embodiment). However, the projections 204 could have a low profile. In addition, by positioning the projections 204 near the center of the contact face 202 as opposed to being close to the edge of the contact face 202, the risk of unintended contact could be reduced.

### Third Embodiment

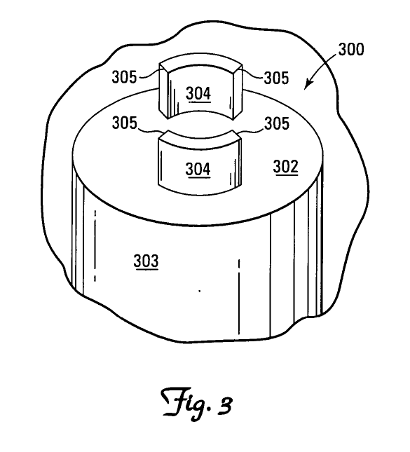

FIG. 3 shows a partial view of a membrane perforator 300 according to a third embodiment of the invention. The projections 304 shown here generally resemble those discussed in relation to FIGS. 1A-1G. In this embodiment, however, the walls of the projection 304 are curved and the sides of the walls are straight. An advantage of projections 304 such as these are that the edges 305 could cause less harm. However, for some applications, the membrane perforator 300 could require more rotation in order the create the shearing forces necessary to break the membrane (not shown in relation to this embodiment).

### Fourth Embodiment

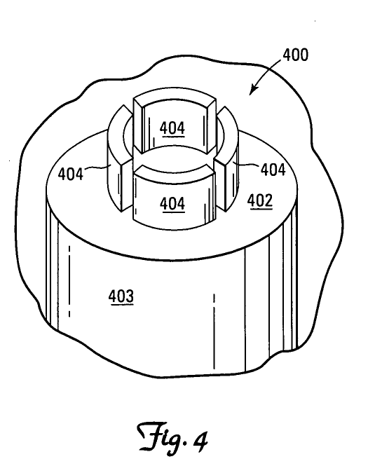

FIG. 4 shows a partial view of a membrane perforator 400 according to a fourth embodiment of the invention. The projections 404 shown here generally resemble those discussed in relation to FIG. 3 except the contact face 402 has four projections 404. Such a membrane perforator 400 might be particularly useful for piercing thinner membranes (not shown in relation to this embodiment).

### Fifth Embodiment

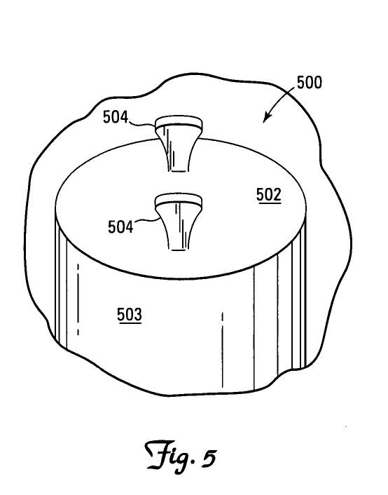

FIG. 5 shows a partial view of a membrane perforator 500 according to a fifth embodiment of the invention. The two projections 504 shown here have curved top edges. Such a configuration could reduce the potential for injury. However, such a configuration could in some applications require more downward pressure parallel to the longitudinal axis of the membrane perforator 500 in order to engage the projections 504 and pierce the membrane (not shown in relation to this embodiment).

### Sixth Embodiment

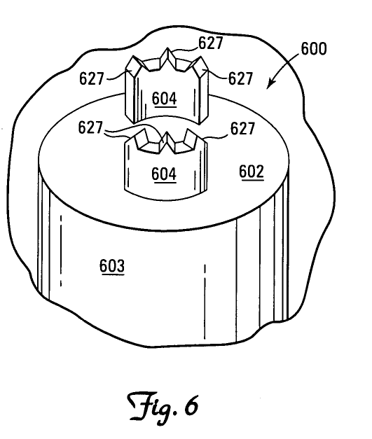

FIG. 6 shows a partial view of a membrane perforator 600 according to a sixth embodiment of the invention. The two projections 604 shown here have curved walls with small serrations 627 on the top of the projections 604. These serrations 627 could serve a number of purposes. First, the serrations 627 could help grip the amniotic membrane (not shown in relation to this embodiment) and make the shearing of the membrane (not shown in relation to this embodiment) more efficient. Second, the serrations 627 could pierce a thinner membrane when downward pressure parallel to the longitudinal axis of the membrane perforator 600 is applied (not shown in relation to this embodiment). Third, the serrations 627 could snag the membrane when the membrane perforator 600 is held at an angle to the membrane surface (not shown in relation to this embodiment).

### Seventh Embodiment

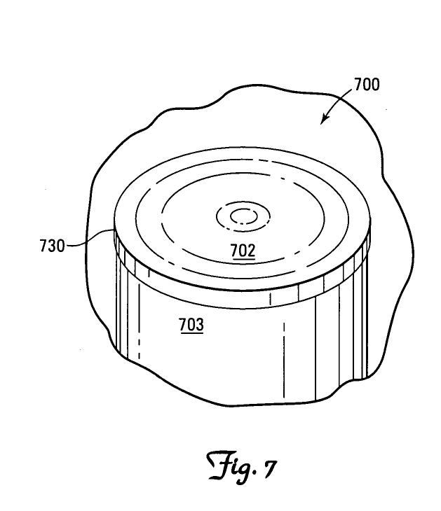

FIG. 7 shows a partial view of a membrane perforator 700 according to a seventh embodiment of the invention. In this embodiment the distal end 703 of the membrane perforator 700 has a cap 730 made of a material different from the distal end 703. For example, the cap 730 could be made of a rubber material. Such a material could be sufficiently tactile to grip the membrane (not shown in FIG. 7) when the membrane perforator 700 is rotated or when a pushing or dragging action is used (not shown in relation to this embodiment). In addition, gripping features such as a tread could be employed to ensure better traction between the contact face 702 and the membrane (not shown in FIG. 7).

### Eighth Embodiment

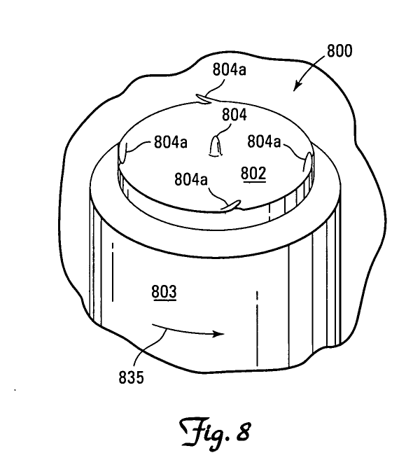

FIG. 8 shows a partial view of a membrane perforator 800 according to an eighth embodiment of the invention. In this embodiment the distal end 803 of the membrane perforator 800 has two kinds of projections. A center projection 804 and four cutter projections 804a. The membrane perforator 800 shown in FIG. 8 could work as follows. A user could position the membrane perforator 800 in the desired position and rotate the membrane perforator 800 in a counter-clockwise direction 835. The membrane perforator 800 would rotate on the center projection 804. As the membrane perforator 800 rotated in the counter-clockwise direction 835, the cutter projections 804a would cut into the membrane (not shown in FIG. 8) and, with sufficient rotation, perforate it.

Modifications

The invention described in this specification encompasses numerous modifications including membrane perforators made of different sizes, shapes, and materials and configured in different ways than discussed above.

Many factors may influence the size and shape of the membrane perforator and its features. For example, for some applications it may be desirable to have a shaft of a different length than described above. It could be desirable to have shafts of a different shape such as an octagonal shape similar to the shaft of a pencil. Finally, depending on the thickness of the membrane to be perforated, it may be desirable to have projections that are of different sizes or shapes than those described above—for example, shorter than those described above. Such aspects of membrane perforators may require modifications in the size and shape of the membrane perforators discussed above in order for the membrane perforator to function as desired. Nonetheless, such changes would be within the scope of the invention.

The membrane perforators discussed above could be made of many different materials. For example, the membrane perforator could be made of various materials including plastic, metal, cellulose based materials, glass or ceramic, or combinations of these materials. The shape of the perforator could be created using many techniques such as molding, forming, or cutting. Such changes would be within the scope of the invention.

As can be seen from the disclosure provided herein, a wide variety of differently sized, shaped and configured projections may be provided on the shaft to achieve the desired function of quickly, easily and safely perforating a biological membrane.

The present invention should not be considered limited to the particular examples or embodiments described above, but rather should be understood to cover all aspects of the invention as fairly set out in the claims arising from this application. For example, while suitable sizes, materials, packaging and the like have been disclosed in the above discussion, it should be appreciated that these are provided by way of example and not of limitation as a number of other sizes, materials, packaging, and so forth may be used without departing from the invention. Various modifications as well as numerous structures to which the present invention may be applicable will be readily apparent to those of skill in the art to which the present invention is directed upon review of the present specifications. The claims which arise from this application are intended to cover such modifications and structures.

We claim:

1. A method of perforating a biological membrane, comprising:
(a) obtaining a medical device, including at least:
(i) a longitudinally elongated shaft defining a longitudinal axis and having a proximal longitudinal end and a distal longitudinal end, and
(ii) a blunt tipped contact face disposed at the distal end of the shaft effective for perforating a biological membrane when the contact face is pressed against a biological membrane and rotated about the longitudinal axis, and
(b) concurrently pressing the contact face against a biological membrane and rotating the shaft about the longitudinal axis so as to effect rotation of the contact face and the biological membrane about a longitudinal centerpoint on the contact face until the membrane is perforated.

2. The method of claim 1 wherein the contact face includes at least one longitudinally extending projection configured and arranged to tear a biological membrane when the projection is pressed against a biological membrane and the shaft is rotated about the longitudinal axis.

3. The method of claim 1 wherein the contact face has a high coefficient of friction sufficient to effect perforation of a biological membrane when the contact face is pressed against a biological membrane and the shaft is rotated about the longitudinal axis.

4. The method of claim 2 wherein the projection has a high coefficient of friction sufficient to effect perforation of a biological membrane when the contact face is pressed against a biological membrane and the shaft rotated about the longitudinal axis.

5. The method of claim 2 wherein (A) pressing the contact face against a biological membrane and rotating the contact face about the longitudinal centerpoint on the contact face creates raised spiral arms in the membrane, and (B) the projection has a sharp side edge configured and arranged to cut into one of the raised spiral arms upon continued rotation of the contact face about the longitudinal centerpoint.

6. The method of claim 1 wherein the shaft is flexible.

7. The method of claim 1 wherein the shaft has a longitudinal length of about 10 inches to about 12 inches.

8. The method of claim 1 wherein the biological membrane is an amniotic membrane.

9. The method of claim 2 wherein the contact face includes a plurality of radially spaced longitudinally extending blunt tipped projections.

10. The method of claim 4 wherein the contact face includes a plurality of radially spaced longitudinally extending blunt tipped projections.

11. The method of claim 5 wherein the contact face includes a plurality of radially spaced longitudinally extending blunt tipped projections.
